(19) United States
(12) Patent Application Publication
(10) Pub. No.: US 2010/0184629 A1
(43) Pub. Date: Jul. 22, 2010

(54) CELL TRAY SYSTEMS AND METHODS

(75) Inventors: Maile Giffin, Kailua, HI (US); Ray Alley, Honolulu, HI (US)

Correspondence Address: Convergent Law Group LLP, P.O. BOX 1329, MOUNTAIN VIEW, CA, 94042, US

(73) Assignee: NANOPOINT, INC., Honolulu, HI (US)

(21) Appl. No.: 12/602,980

(22) PCT Filed: Jun. 5, 2008
(86) PCT No.: PCT/US08/65912

§ 371 Date: Dec. 3, 2009

Related U.S. Application Data
(60) Provisional application No. 60/942,075, filed on Jun. 5, 2007.

(51) Int. Cl. C40B 60/00; C12M 1/00

(52) U.S. Cl. 506/33; 435/307.1

(57) ABSTRACT

A method and system for describing a cell tray are described. The method and system includes providing a first layer and a second layer. The first layer is of an optically transparent substrate material. The second layer is on top of the first layer, the second layer includes a plurality of cell wells, each of the plurality of cell wells being formed by penetrating the second layer to a preselected depth.

### BACKGROUND OF THE INVENTION

Many experimental procedures depend on being able to grow and maintain collections of cells in an arrangement wherein they may be further analyzed. Collections of cells may be studied visually or optically, or may be subject to other interventions. There is particular utility to studying cells that are spatially distributed in an ordered arrangement that is adapted for a variety of individual cell-based assays.

This general principle is illustrated, for example, in commonly-assigned U.S. Pat. No. 7,190,449. The '449 patent describes a two-dimensioned array of cells that includes a set of precise, equally spaced rectangular cubicles or cylindrical silos containing life-support medium, particularly suited for use with a specific high-resolution instrument for analyzing and comparing molecular characteristics of cells. A need exists in the art, however, for a two-dimensioned array of cells suitable for use with a variety of analytic instruments, including imaging systems.

### BRIEF SUMMARY OF THE INVENTION

A method and system for describing a cell tray are described. The method and system includes providing a first layer and a second layer. The first layer is of an optically transparent substrate material. The second layer is on top of the first layer, the second layer includes a plurality of cell wells. Each of the plurality of cell wells being formed by penetrating the second layer to a preselected depth. In some embodiments, the systems and methods may include providing a platform for bioassays and biology imaging analysis. In embodiments, cell trays are disclosed having ordered arrays of micron-dimensioned cell wells for supporting multiple individual cells and presenting them for simultaneous analysis. In embodiments, a cell tray may permit an ordered array of biological material to be processed in parallel. In embodiments, a cell tray bearing multiple micron-dimensioned cell wells may be fabricated in the same shape and size as a conventional microscope slide so that it may be used, for example, with conventional analytic tools.

According to the method and system disclosed herein, cell trays having improved utility may be provided.

### BRIEF DESCRIPTION OF SEVERAL VIEWS OF THE DRAWINGS

FIG. 1 shows a schematic top view of an exemplary embodiment of a cell tray.

FIG. 2 shows, in more detail, an exemplary of one arrangement of microfluidic channels from FIG. 1.

FIGS. 3A-H show exemplary embodiments of possible patterns of fiducials useful with cell trays.

FIG. 4 shows a schematic of an exemplary embodiment of a cell tray with fiducials located thereon.

FIG. 5 shows an exemplary embodiment of a cell tray having a registration key.

FIG. 6 shows an exemplary embodiment of a printed micro-array that may be used to form cell immobilization zones on a cell tray.

FIG. 7 shows an exemplary embodiment of a ceramic add-on for forming cell wells.

FIG. 8 shows schematically the steps of an exemplary embodiment of a process for forming cell wells in a cell tray.

FIGS. 9A and B show elements of an exemplary embodiment of a mask for constructing a cell tray.

FIGS. 10A and B show a schematic top view of an exemplary embodiment of a cell tray displaying an arrangement of wells and microfluidic channels.

FIG. 11 shows an exemplary embodiment of a cell tray having no microfluidic channels.

FIGS. 12A and B show an exemplary embodiment of a cell tray with a detail of a well array system.

FIGS. 13A-D shows exemplary embodiments of a cell tray holder.

### DETAILED DESCRIPTION OF THE INVENTION

The method and system relate to a method and system for providing and utilizing cell trays. The following description is presented to enable one of ordinary skill in the art to make and use the invention and is provided in the context of a patent application and its requirements. Various modifications to the embodiments and the generic principles and features described herein will be readily apparent to those skilled in the art. Thus, the method and system are not intended to be limited to the embodiments shown, but is to be accorded the widest scope consistent with the principles and features described herein. For example, the method and system are mainly described in terms of particular systems provided in particular implementations. However, one of ordinary skill in the art will readily recognize that this method and system will operate effectively in other implementations. The method and system will also be described in the context of particular methods having certain steps. However, the method and system operate effectively for other methods having different and/or additional steps not inconsistent with the method and system. The method and system are described in the context of particular measurements. However, one of ordinary skill in the art will also recognize that such measurements are within expected tolerances.

Described herein are embodiments of a miniaturized microtiter plate termed a “cell tray.” The cell tray contains a plurality of cell holders, which may be configured as volume-containing cell wells or as cell immobilization zones. A cell well contains a defined fluid volume, and is generally configured as a depression or as a cavity in a solid substrate. A cell immobilization zone is an area on a solid substrate treated with a substance that immobilizes or otherwise holds cells without confining them to a volumetric space, for example by attaching the cells to an adherent material or by reacting a surface of the cells to a reagent that affixes them through the contact.

In some embodiments, a cell tray may contain an array of cell wells formed by either etching into a base substrate or by bonding two or more material components together in which the top layer has bored through holes that reach the bottom substrate. The liquid volumes used in these wells are generally on the order of pico-liter up to micro-liters. In some embodiments, the cell tray contains an assortment of cell wells of preselected sizes, depending on the research applications for which it is used. For example, the cell tray can be used as a biosensor in which wells can be preloaded with biological agent or markers and cells or other biological material can be added after the fact. In one embodiment, a cell tray may be prespotted with a number of wells containing markers for different diseases or chemicals. In such an embodiment, the cell tray might be used in a clinical environment to identify diseases, or in the field to identify biohazards or chemical hazards. A pre-spotted cell tray may be sealed in a sterile packet for microbiological or medical applications, for example, for uses for a specific patient. In a single-patient application, a sample from the patient may be applied across an array of wells, where each well contains a marker for a disease state or for a pathological organism. In some embodiments, the cell tray is dimensionally adapted for reading with a laboratory-scale fluorescence scanner or with a portable fluorescence scanner. A cell tray so shaped and sized may contain arrays that hold reagents capable of fluorescence, for example when the reagent is contacted by a particular chemical or biological specimen. Fluorescence in certain wells and not in others can be indicative of disease states. Other uses for the cell tray will be apparent to one of ordinary skill in the art.

In some embodiments, a cell tray according to these systems and methods includes an optically transparent glass substrate having a two-dimensional array of rectangular, cylindrical or otherwise regularly shaped wells etched into its surface. In some embodiments, the cell tray may be formed from a substrate such as borosilicate or fused silica, and may be sized to match the size of a standard microscope slide. For example, a cell tray may have dimensions of 76.2×25.4 mm (length×width) with thickness of 1.1 mm. Other dimensions of length, width and thickness can be readily envisioned. The aforesaid dimensions are particularly suitable for microscopic applications, but are not required for the method and system described herein. Sizes for cell wells generally range from widths of 50 μm to 300 μm for cell trays used for biological applications, although smaller or larger sizes may be appropriate for other uses. In some embodiments, well sizes of 50 μm, 100 μm, 200 μm and 300 μm may be formed. Well sizes may be selected for specific applications, so that wells are capable of holding a single cell, a few cells, or any number of cells as would be appropriate for a particular experiment or set of experiments.

In some embodiments, there may be a high density of cell wells on the cell tray, for example, 10,000 per slide, optionally arranged in subunits or arrays as a particular analytic application may require. In other embodiments, cell trays may be fabricated containing wells of larger size, or wells that are all the same size. For instance, wells may be larger than the 50-100 μm size. In an embodiment, the cell tray contains wells 200 μm in diameter. In an embodiment, the cell tray contains wells 300 μm in size. The cell tray can contain cell wells of other sizes, in keeping with the needs of users of the device, e.g., researchers. Wells may be round, square, rectangular, or any other shape that would be useful for specific applications.

It is understood that the cell tray might have any number of cell wells. In an embodiment, the cell tray can have 32 addressable arrays of cell wells, each array containing sixteen 300 μm wells. Other configurations of wells and well units are apparent to those of ordinary skill in the art.

A microfluidics system may also be etched into the substrate, interconnecting some or all of the cell wells. The microfluidics system may interface with a plurality of microfluidics channels that provide fluid influx and/or efflux to the microfluidics system and thence to the cell wells. In some embodiments, patterns of microfluidics may be formed, consistent with particular analytic applications. It would be understood by one of ordinary skill in the art that a microfluidics system, operating for example by capillary action, may conduct fluid between and among cell wells. In some embodiments, markers can be used to identify components of the microfluidic channel system. For example, metallic markers can be used to identify the inflow or outflow channels, making them easier for a user to see them when manually pipetting fluids into or out of such channels. It would be understood by those of ordinary skill in the art that the influx or efflux of fluid from the cell wells via the microfluidics channel may be sampled or otherwise monitored for relevant attributes, e.g., flow rate, temperature, pH, the presence or absence of various metabolites or other chemical byproducts, and the like.

Various arrangements of cell wells on the cell tray may be provided, for example with groupings of wells by size, or with ordered arrays of wells of different sizes or different shapes. A number of discrete regions having cell well arrangements may be configured on a single cell tray. For example, a cell tray might contain fourteen discrete regions, or more or fewer regions as desired by users of the device, e.g., researchers. In an embodiment, 8 regions of cell wells are arranged on a cell tray, each well being square in shape. Other configurations of cell wells in regions on a cell tray can be readily envisioned.

In some embodiments, a cell tray may be fabricated without interconnecting fluid channels. Such a cell tray may be suitable for spotting applications, either by automated loaders (e.g., liquid handling or spotting machines) or by manual techniques in a research laboratory. For example, for use with spotting machines, a cell tray with individual square wells 700 μm in width may be constructed, so that the wells are appropriate for the fluid volumes that such machines dispense. For this application, cell wells may be 20 μm in depth in some embodiments, although well depth may be adjusted to meet the needs of the particular application. Well depths of several hundred microns are consistent with the disclosed systems and methods.

In an embodiment, a cell tray can be fabricated using multiple layers of an optically appropriate material that are then bonded together. For example, a photosensitive glass such as Foturan® can be used for the layers, or a mixture of glass and silicon can be used. The base layer may have some fiducial markings on it, as described in more detail below. For a representative embodiment, chrome can be used for the fiducial markings, but a variety of other metals or marking systems may be used, such as etched marks in the substrate. Using a photosensitive material like Foturan® allows fiducial markings to be inscribed within a layer by exposing masked areas to light. Desirably, the bottom layer is optically clear, so either glass or an optically clear plastic can also be used for this layer. The bottom layer may contain wells or depressions, but it preferably does not contain through-holes.

The middle layer may be constructed with through-holes in it that form the wells, or that correspond to wells or depressions in the bottom layer. If a microfluidics system and/or microfluidic channels are desired, they can be etched in this layer as well. Through-holes in this layer may also be located over any fiducial markings on the base substrate. Alternatively, a fiducial pattern may be inscribed in the middle layer.

The top layer may contain through-holes that match the through-holes in the middle layer, permitting visualization of the fiducials, for example. A single, large hole in the top layer may be positioned to expose an array of underlying cell wells. Any configuration of geometries may be envisioned, so that the hole pattern in the top layer interfaces usefully with the hole pattern in the middle layer. The top layer can have a separate set of through-holes that extend through the middle layer to the bottom substrate, useful, for example, as air vents.

In some embodiments, a number of other materials might be used for the layered structure of a cell tray. In some embodiments, all layers can be fabricated from Foturan®. In other embodiments, certain layers may be made from other materials, including but not limited to photoetched metals such as Kovar™ or Invar, appropriate ceramics or polymers, as long as the thermal coefficient of expansion for the selected material is compatible with the thermal coefficient of expansion of the other material layers.

In some alternative embodiments, a cell tray may be fabricated from a single substrate with an adhesive or pressure sensitive gasket to isolate regions on the cell tray, so that cutout regions on the gasket align with regions on the cell tray. Using this design, a cell tray can be fabricated to minimize evaporation issues, because a larger volume of fluid can be added to each region. The gasket arrangement can effectively isolate cell well regions so that there is no spillover of fluid from one region to another.

In some embodiments, additional features can be added to or embedded in the cell tray substrate to provide specific functionalities. For example, electronic components and/or sensors may permit detection of certain conditions within the cell wells, or might allow ongoing monitoring of biological, fluid, cellular or molecular changes. Active photonic devices, such as a pH detector, might be integrated into a cell tray layer or added on to the cell tray. Lasers and other detectors are known in the art to be integratable onto chips for diagnostic or measuring purposes. Such devices may be added to the cell tray fabrication using no more than routine experimentation. In other embodiments, materials or coatings may be added to the cell tray as separate layers or as coatings on the existing layers. Such additional layers or coatings may provide additional functionalities, acting for example to filter for light, or to change properties of incident light such as its phase or its polarization, or to shape light by focusing it or diverging it. In some embodiments, the material of the substrate itself can be selected to match a particular range of the electromagnetic spectrum.

In some embodiments, large reservoirs may be formed in the glass substrate of the cell tray to provide specific functionality. The volume of such reservoirs may be substantial relative to the volume, for example, of an individual cell well. Reservoirs may be connected to other cell wells with microfluidic channels, or interconnected to other reservoirs on the cell tray, or unconnected and stand-alone. Reservoirs may be used to contain, for example, cell culture media or other fluid, reagents, extra cells or other biological material.

Cell trays fabricated in accordance with these systems and methods may be used in conjunction with a cell tray holder that holds one or more cell trays in an easily accessible configuration. In an embodiment, a cell tray holder can hold three cell trays in a space usually occupied by a standard well plate, but other configurations of cell tray holders may hold other numbers of cell trays. In some embodiments, the cell tray holder provides an interface between one or more cell trays and existing liquid handling machines that may spot fluid or other agents into microtiter plates.

In some embodiments, alignment fiducials may be embossed upon the substrate to allow the cell tray to be calibrated. Fiducials, when used as a calibration point, allow automated and manual focusing of the cell tray. Fiducials may be arranged in primary and secondary patterns. Fiducials may be used as alignment tools themselves, or may be used to point to other focusing fiducials. Besides visual aids for alignment, fiducials of known length may be used to calibrate the field of view of an optical system. Fiducials may also be used as security icons, where a particular pattern is used as a code for a particular product.

In some embodiments, a software system may interface with the arrangement of fiducials so that a user may calibrate the cell tray on a microscope platform. Thus, the cell tray may be auto-calibrated. In some embodiments, a user may employ the fiducials to scan various parts of the cell tray using, for example, a microscope. The user might also use the fiducials to return to a specific cell well while examining the plurality of wells on the cell tray. In some embodiments, a marker such as a notched corner may be provided for registration.

In some embodiments, a cell tray may be fabricated by printing a two-dimensional pattern on top of an optical quality substrate to form cell-adherent zones on the cell tray. Cell-adherent zones perform the function of cell wells without need for etching or any other invasive process. In some embodiments, a printed square may be designed from a material that attracts cells or causes biological material to adhere to it, for example, poly-L-lysine, collagen, and the like. In an exemplary embodiment of a printing arrangement, areas adjacent to cell-adherent zones may be “blanks” that are untreated or are treated with a hydrophobic material, so that cells or other biological material are directed to the cell-adherent zones.

In some embodiments, a cell tray may be fabricated by adding a curable ceramic or the like to a base material, for example a glass substrate. Such added material may be hardened, through chemical contact, light exposure or the like. In some embodiments, an optical quality glass substance may be layered with ceramic or the other material, and a mask placed over it. If the mask and the ceramic are then exposed to UV light, the ceramic not covered by the mask hardens. The remaining “undeveloped” ceramic may be removed with a solvent, leaving eroded areas where the ceramic has been removed from the cured ceramic layer. In some embodiments, the eroded area extends to the glass of the cell tray substrate, so that the bottom of the eroded area is glass. Each eroded area then may constitute a cell well, according to the systems and methods described herein. In some embodiments, the mask used for producing the pattern of eroded areas may have a checkerboard pattern or any other pattern that will produce a useful arrangement of cell wells.

In some embodiments, cell trays may be formed by depositing a layer of metal, for example chrome or aluminum, on the optically transparent glass substrate. After forming a pattern in the metal with photolithography technique(s), the metal is etched to create the desired pattern in the substrate. In some embodiments, the etch depth of the cell wells is at 20 μm. In embodiments, the mask may be formed to result in 14 blocks or regions on the substrate, with each region containing a plurality of cell wells having sizes ranging from 50 μm to 300 μm. For example, the regions containing cell wells may bear a grouping of wells of a particular dimension, for example 50 μm, 100 μm, 200 μm and 300 μm size wells arranged to form a pattern in the substrate, with a microfluidic system interconnecting wells in the same region. Wells may be etched at the same depth in a group, for example 20 μm in depth. Alternatively, wells in different groups, or different wells within a single group, may be etched with different depths. Combinations of etching depths may be designed for specific applications, as would be understood by one of ordinary skill in the art. In some embodiments, a variety of experiments may be run simultaneously on one cell tray. For example, 14 different experiments may be run simultaneously on a cell tray having 14 arrays or regions, as described above.

It would be understood by those of ordinary skill in the art that many other fabrication methods may be used to form cell wells on the cell trays described herein, for example, photolithography, laser etching, deep reactive ion etching (DRIE) or ceramic add-on methods. A cell tray may be formed from a single material of an optically transparent glass, for example, or it may be formed from layers of the same or different materials bonded or otherwise joined together.

For cell trays having no microfluidic system or channels, other fabrication methods might be used. For example, one embodiment of a method might use a plain glass slide and etch the wells into the glass. In other embodiments, the tray may be formed as a composite of two or more components that are bonded together. In such embodiments, the base substrate may be glass or any other transparent material such as plastic (e.g., cyclic olefin copolymers such as Topas® COC, XEONOR, polymethylmethacrylate, and other material(s)). In a preferred embodiment, the material should be suitable for biological applications. As would be understood by those of ordinary skill in the art, various parameters of a polymeric material must be considered to determine its suitability for biological applications, e.g., the degas number for the material, or its ability to withstand cleaning reagents and/or autoclaving.

The upper layer of the cell tray (which is bonded to the base) may be made of a number of materials. For compatible materials such as borosilicate with silicon, anodical bonding may be appropriate for certain applications. For rubber or plastic to be bonded to glass or similar substrates, epoxy or other adhesives may be used. Another method for bonding substrates is to place thin film material or thermal film between the substrates. Heat is then applied to solidify the bond. This bonding process, known as diffusion bonding, is appropriate for many polymeric materials. When using a bonded cell tray made from two or more components, the wells may be produced from through holes made in the upper substrate, and the base substrate (glass or other material) may not require any etching.

While certain of the fabrication techniques described herein may be familiar to those of ordinary skill in micromachining, these systems and methods are specifically adapted for producing a cell support system having a multitude of cell wells of micron-scale dimensions ordered in a regular array. Features of a cell support system in accordance with these systems and methods may be understood in more detail with reference to the drawings.

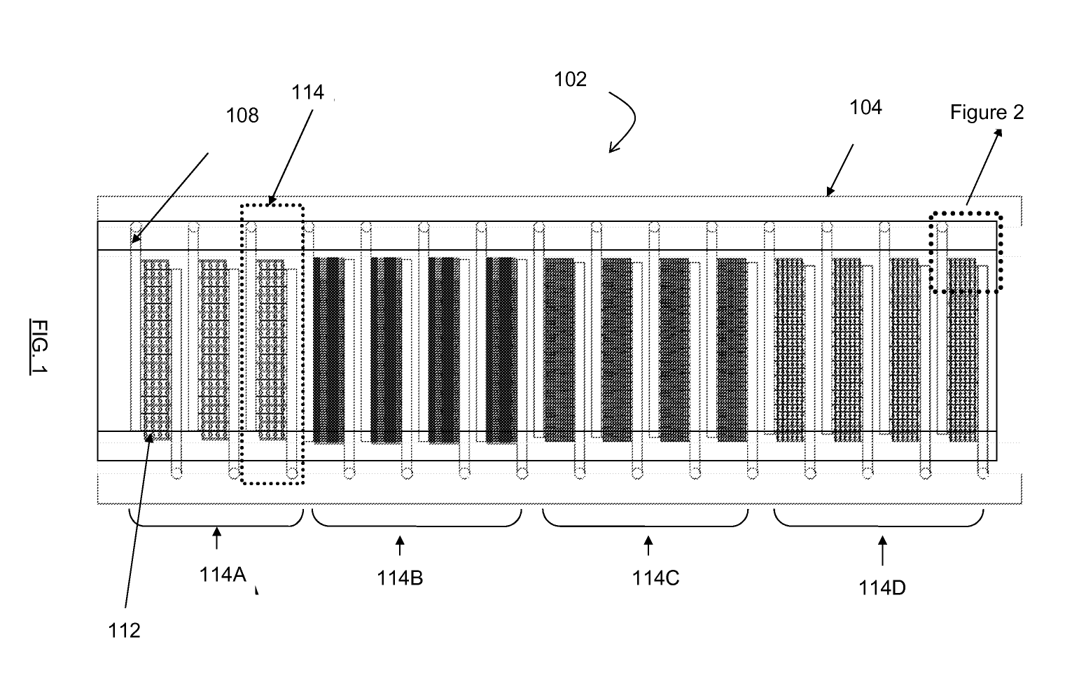

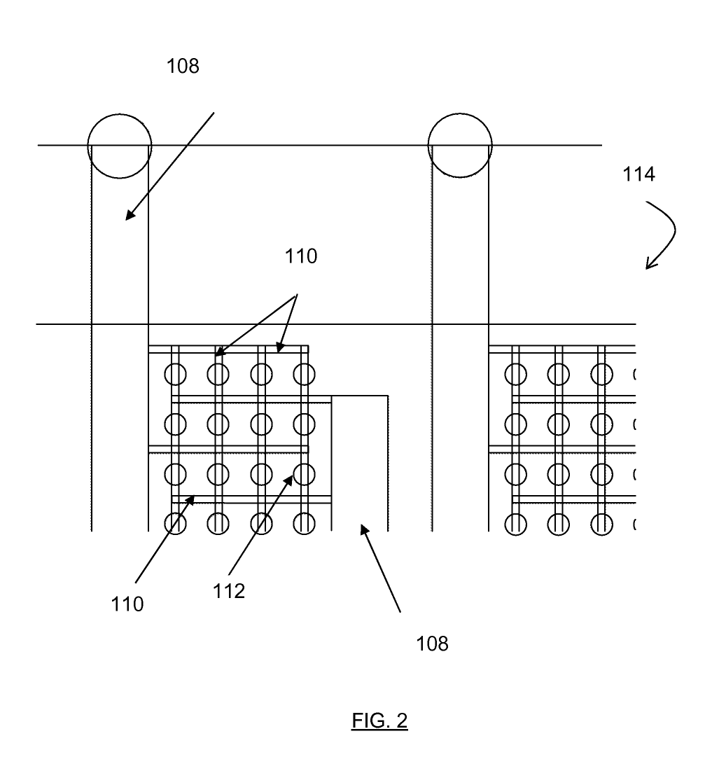

FIG. 1 depicts an exemplary embodiment of a cell tray 102 that may be formed on a substrate block 104 having a plurality of cell wells 112 arranged in arrays 114. In one embodiment, the substrate block may be optically transparent. As depicted in FIG. 1, each array 114 may contain cell wells 112 of a specific size. Large microfluidic channels 108 may provide nutrients or other fluids to an array 114, with smaller microfluidic conduits (shown in more detail in FIG. 2) connecting the large channels 108 to the individual cell wells 112. In the depicted embodiment, sets of arrays 114 are grouped together to form regions 114A, 114B, 114C, 114D, with each region being characterized by cell wells 112 of a specific size. A section of an array 114 is shown in more detail in FIG. 2.

FIG. 2 shows, in more detail, an exemplary embodiment of a section of an array 114 bearing a plurality of cell wells 112 interconnected by a microfluidic system. This figure depicts a large microfluidic channel 108 on the inflow side giving rise to a number of small microfluidic conduits 110, with the small microfluidic conduits 110 each branching to feed an individual cell well 112. A corresponding arrangement is seen on the outflow side, where small microfluidic conduits 110 drain the individual cell wells 112 and transport the drainage fluid to a large microfluidic channel 108.

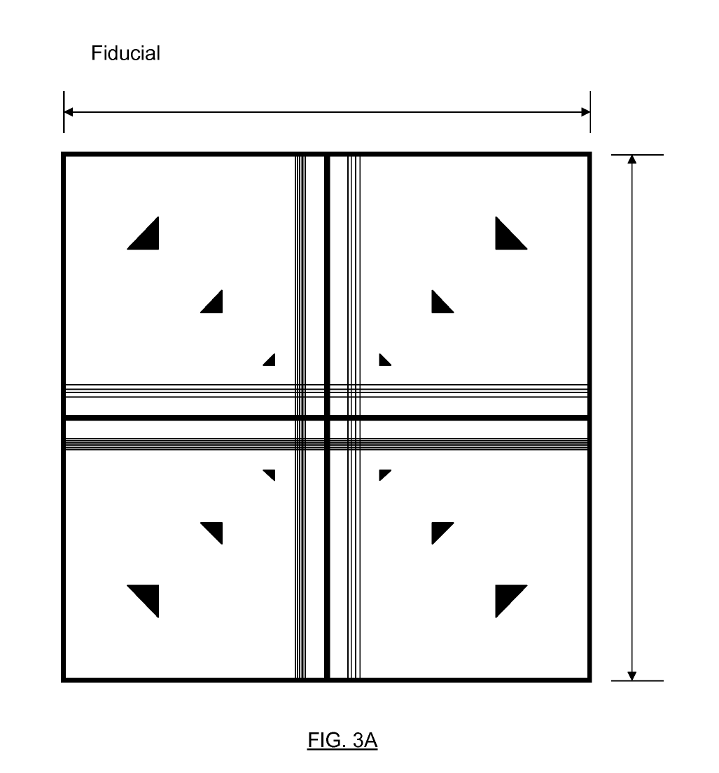

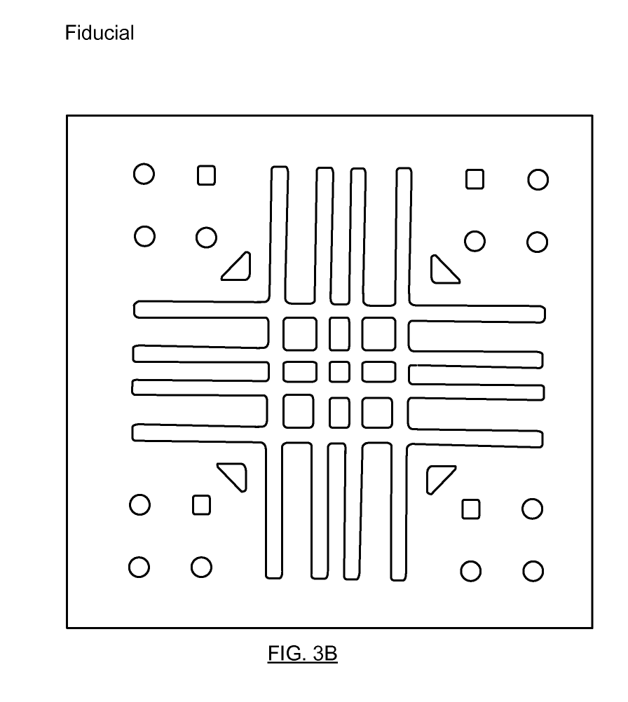

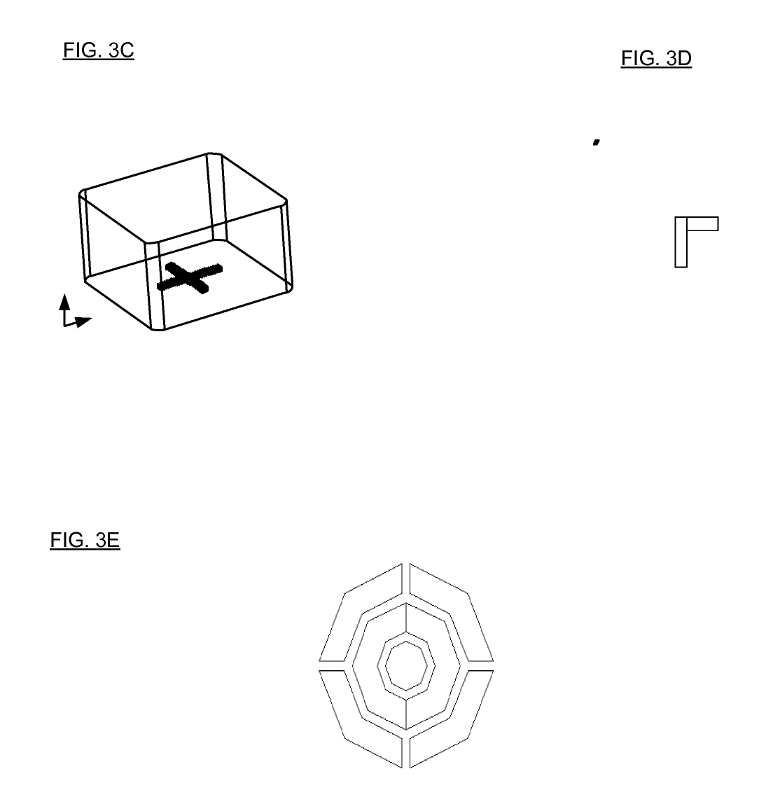

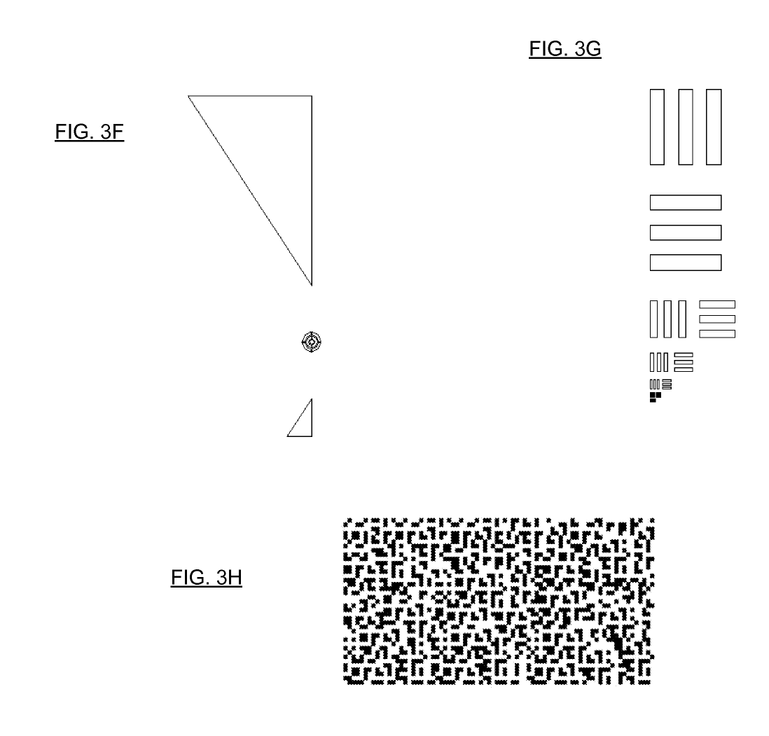

FIG. 3A illustrates an exemplary embodiment of a fiducial that may be positioned on the cell tray. FIG. 3B shows an alternate embodiment of a fiducial that may be positioned on the cell tray. A fiducial system may take a variety of forms, so that it may be recognized, for example by appropriate software for aligning the cell tray. In the depicted embodiments, a complex of intersecting lines has been arranged as a fiducial to enable the recognition software to distinguish the fiducial from other computer-recognizable lines or interfaces on the cell tray. In other embodiments, such as those shown in FIGS. 3C-3H, additional geometric figures may be added for a variety of reasons. For example, additional geometric figures may heighten fiducial recognizability or interface with other recognition features in the computer software.

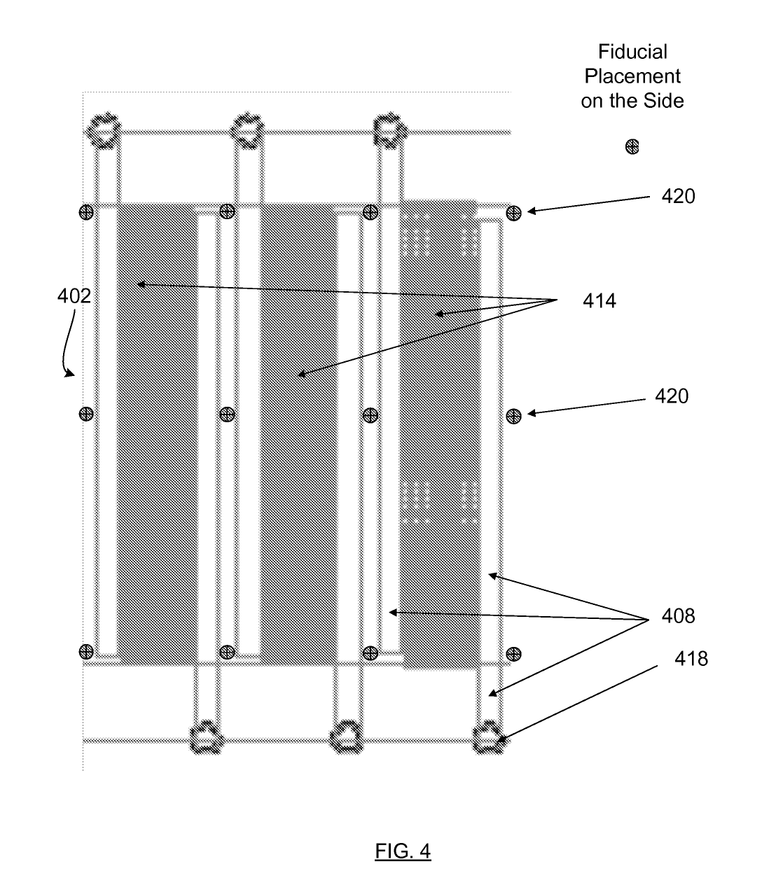

FIG. 4 depicts an exemplary embodiment of how a set of fiducials 420 may be positioned on a section of a cell tray 402. In the illustrated embodiment, showing a number of arrays 414 containing cell wells and microfluidics, fiducials 420 are positioned between two adjacent arrays 414. More specifically the fiducials 420 reside between two large microfluidic channels 408. In this embodiment, a set of visualization markers 418 is shown surrounding the inlets and outlets for the large microfluidic channels 408. In some embodiments, visualization markers 418 such as chrome circles or other markers are placed to designate the location of certain cell tray 402 features to make them more apparent to the naked eye.

In FIG. 5, an exemplary embodiment of a cell tray 102 is shown where the substrate 104 has three beveled corners 150 and one right angle corner 152. The right angle corner 152 may act as a registration key to couple the cell tray 102 to other devices made uniquely for this product, for example, a platform that mounts the cell tray 102 to a microscope stage. As depicted in this figure, the cell tray 102 is made in a size and shape of a typical microscope slide. The beveled edges 150 are included for ease of handling.

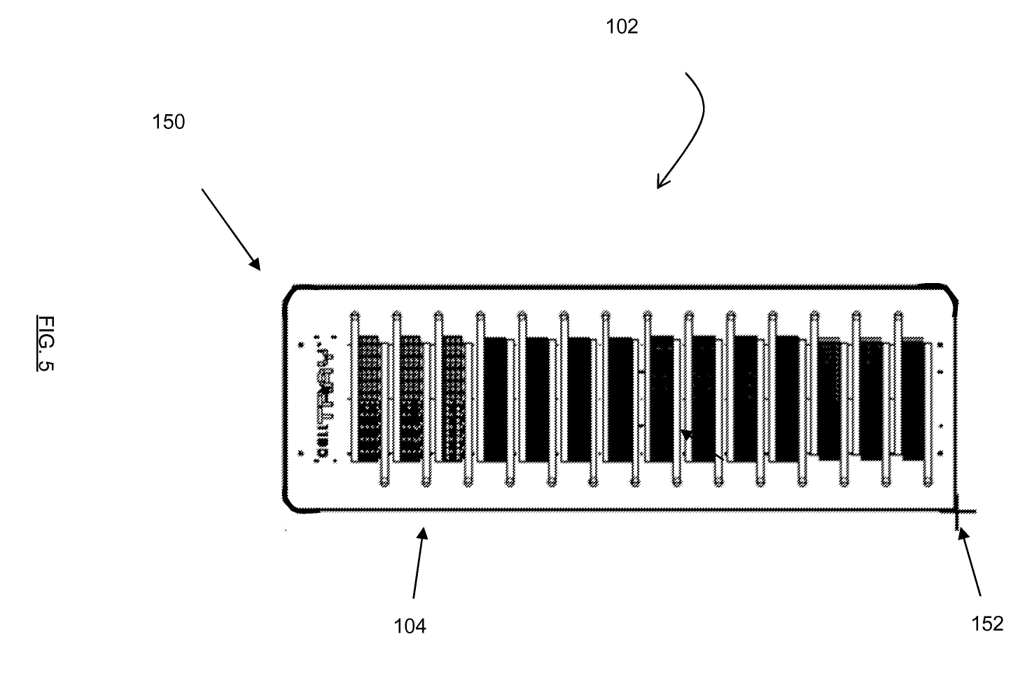

FIG. 6 illustrates, in an exemplary embodiment, the use of a printing method to construct a micro-array of cell immobilization zones, here shown as printed squares 212, on a cell tray 202. In the depicted embodiment, an optical quality substrate 204 may be formed as described above. Printed squares 212 of material on the substrate may be used for specific purposes. For example, these printed squares 212 may form alternating regions of cell attraction and cell nonadherence. It would be understood by those of ordinary skill in the art that any pattern may be formed on the substrate 202 so that cell adherent areas may be arranged in ways that are adapted to a particular application. As would be appreciated by those of ordinary skill in the art, the printed regions need not be squares. It would be appreciated by those of ordinary skill in the art that the printed squares 212 might be positioned flush with the surface, may be indented, may be raised. The depicted cell tray 202 system does not involve any microfluidics, because no fluid transfers between or among the printed squares 212.

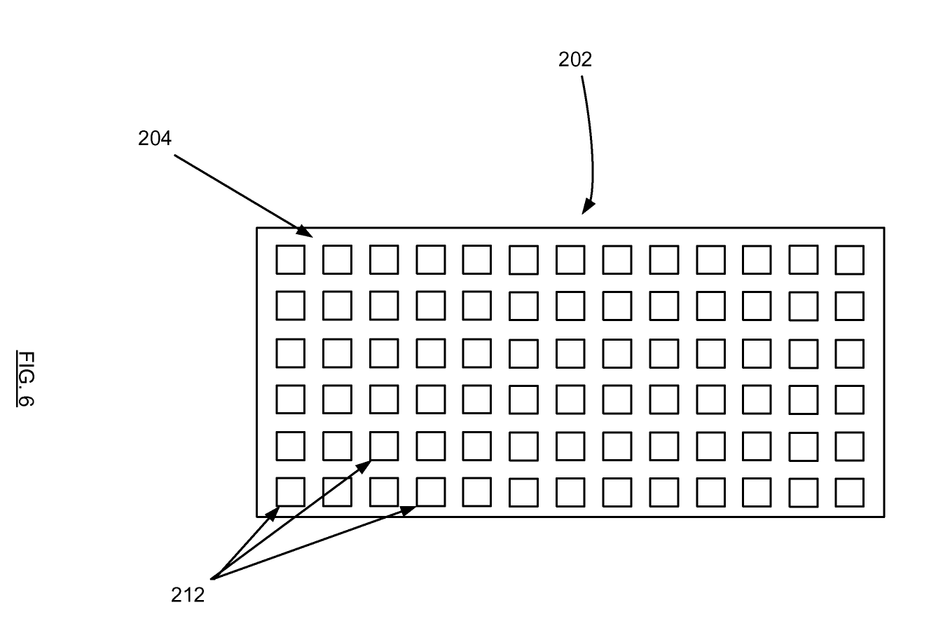

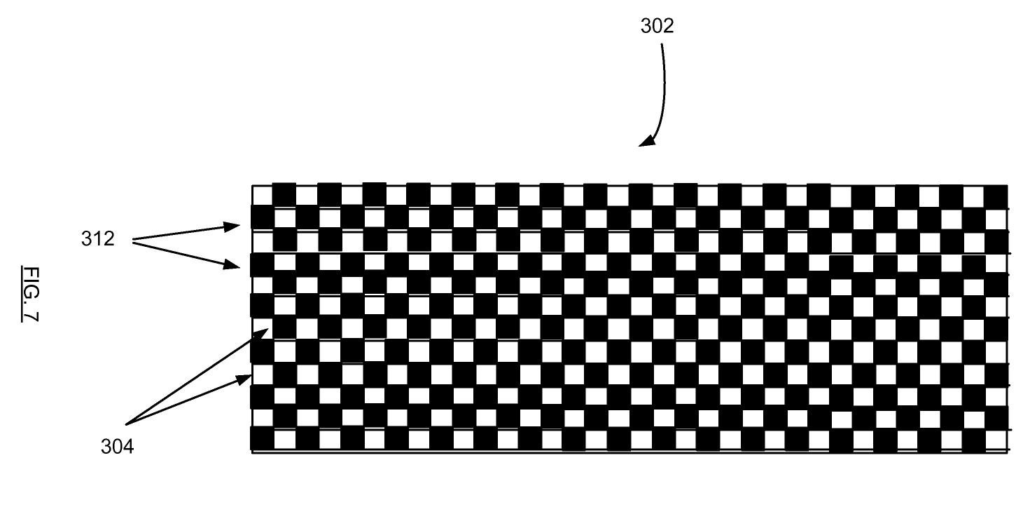

FIG. 7 shows an exemplary embodiment of a pattern for cell wells to be formed on a glass substrate using the ceramic hardening technique described above. In the depicted embodiment, a pattern was formed by using a mask over a ceramic layer (not shown), where the ceramic layer formed the top layer of a cell trade 302. The unmasked areas of the ceramic layer were exposed to a curing agent such as UV light to harden them. The un-hardened (masked) areas of ceramic were then removed using a conventional etching formulation, such as a solvent, leaving a pattern of hardened ceramic elements 312 alternating with wells formed through the ceramic where the underlying glass substrate 304 is exposed. As would be appreciated by those of ordinary skill in the art, an extensive variety of patterns can be designed for a cell tray 302 by using this technique.

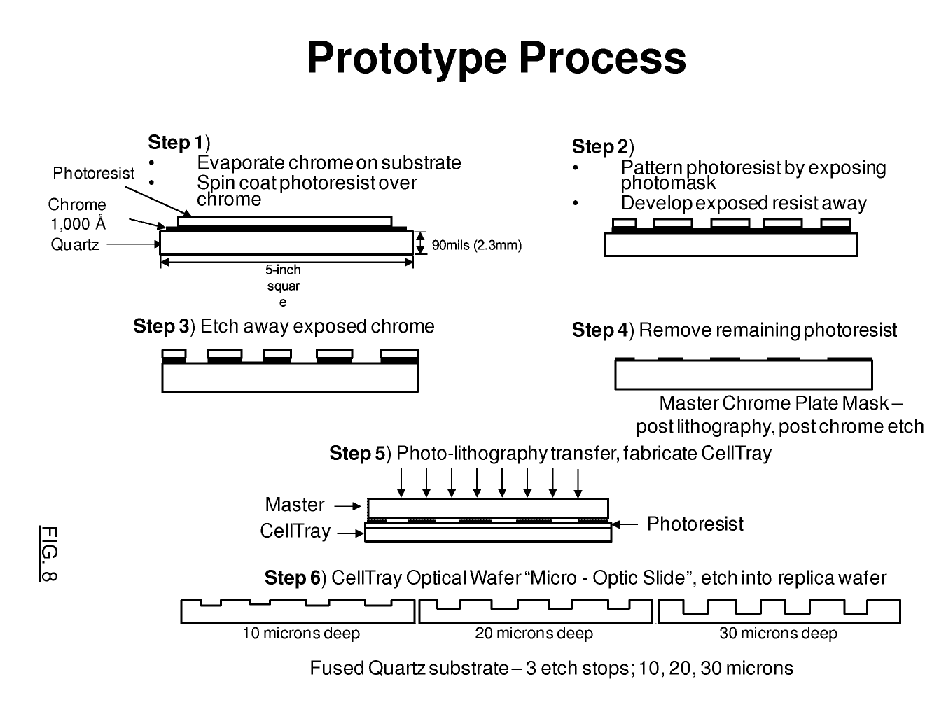

FIG. 8 illustrates schematically an exemplary embodiment of a process for forming an embodiment of a cell tray. Step 1 may involve evaporating a metal like chrome in a layer, for example having a thickness of approximately 1,000 Angstroms, to cover the glass substrate of the cell tray. A photoresist coat may be spun over the chrome. Step 2 may involve placing a photomask over the photoresist to create a pattern, so that the exposed photoresist may be developed away. Step 3 may involve etching away the exposed chrome. In Step 4, the remaining photoresist is removed, so that a master chrome plate mask is produced. In Steps 5 and 6, the substrate for the cell tray is coated with photoresist. The master chrome plate mask having the master image of the device on it may then be placed over the coated substrate. With mask in place, the substrate is then exposed to light, for example high intensity ultraviolet light, which causes the photoresist layer on the substrate to chemically alter.

Once exposed, the substrate is then immersed in a developer solution. Developer solutions are, in some embodiments, aqueous, capable of dissolving away areas of the photoresist that were exposed to light. After successful development, the photoresist is patterned with the master image. After exposure to the developer, the substrate may be baked in an oven or hot plate at temperatures between 100-120 C in order to drive off liquids that may have been absorbed on the substrate, for example, or to crosslink the remaining photoresist. In embodiments, crosslinking the polymer may increase mechanical and chemical stability of the material, allowing it to be used in further substrate processing. As would be understood by those of ordinary skill in the art, the process above is one embodiment of a fabrication process for a cell tray. Other methods of fabrication involving, for example metal masks, photoresists, and etching, may be readily envisioned. Furthermore, as would be understood by skilled artisans, photoresist technologies suitable for use with the present systems and methods may involve either positive or negative photoresists.

Another method that can be used to fabricate the cell tray is Deep Reactive Ion Etching (DRIE). First a very thin layer of metal, chrome or aluminum, is placed on a glass wafer. Secondly, a very thin layer of photoresist is coated on the wafer. The wafer is then exposed to UV light using a mask, which activates the exposed areas of the photoresist imaged by the mask. The areas of exposed photoresist are etched off to the surface of the glass wafer. This leaves the cell tray with all the metal finishing's. Next, another very thin layer of photoresist is coated on the wafer. The wafer is again exposed to light using a second mask. The exposed areas of photoresist become activated and can then be etched to a 20 micron depth, or any desired depth. The wafer is cleaned, diced, and a final product is ready.

As would be understood by one of ordinary skill in the art, other methods of fabrication may be employed for the manufacture of cell trays. For example, laser direct write grayscale photolithography or grayscale projection photolithography may be employed. Electron beam lithography may also be suitable. Other manufacturing methods would be apparent to those of ordinary skill in the art.

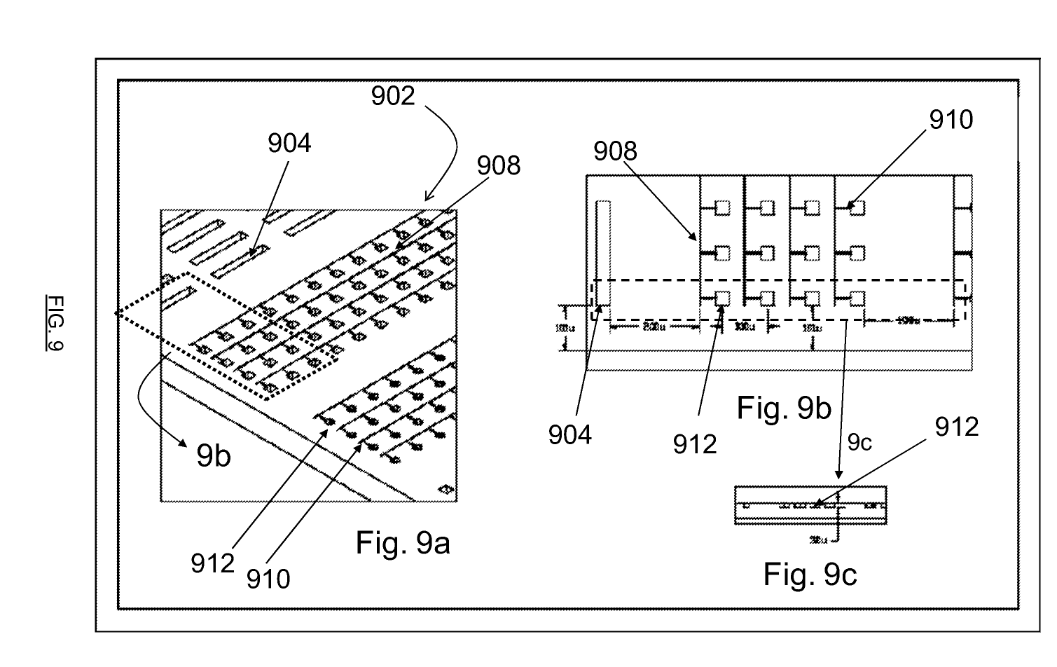

FIG. 9 shows an exemplary embodiment of a cell tray mask 902 that can be used to fabricate the cell wells, microfluidic conduits and channels, reservoirs, and other features of a cell tray. As shown in FIG. 9, the mask contains elements corresponding to reservoirs 904 individual cell wells 912, small branch microfluidic conduits 910, and small stem microfluidic conduits 908. FIG. 9A shows a section of the mask 902 with all these mask elements visible. FIG. 9B shows a section of the mask 902 of FIG. 9A, with the reservoir element 902, the cell well elements 912, the microfluidic branch conduits 910, and the microfluidic stem conduits 908 all shown in more detail. This figure also shows some of the dimensions involved in the depicted embodiment.

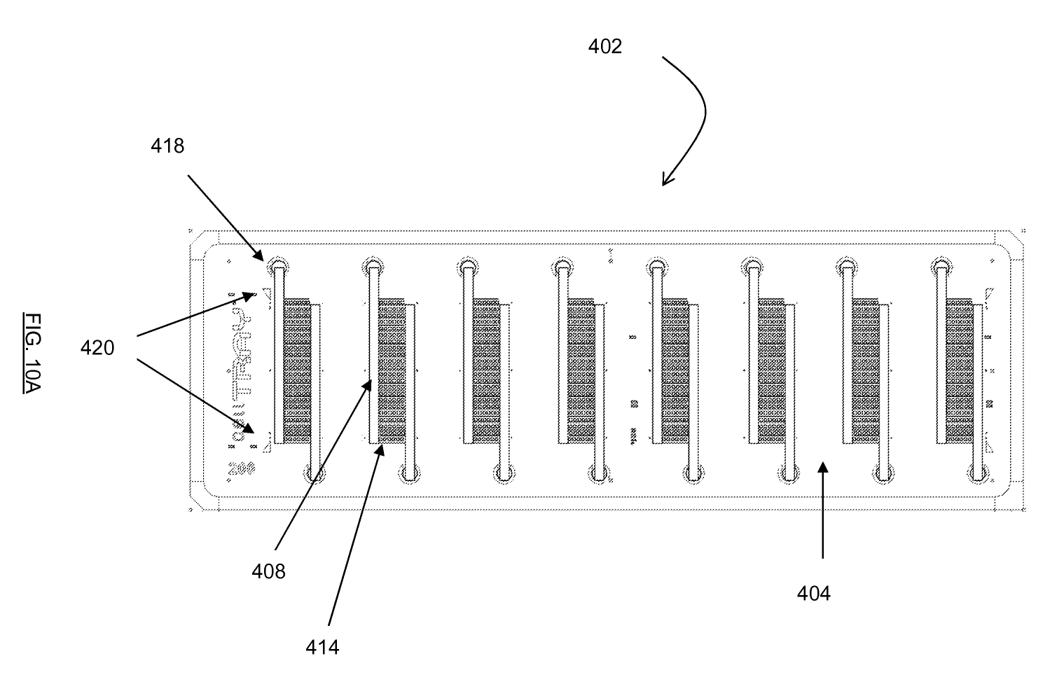

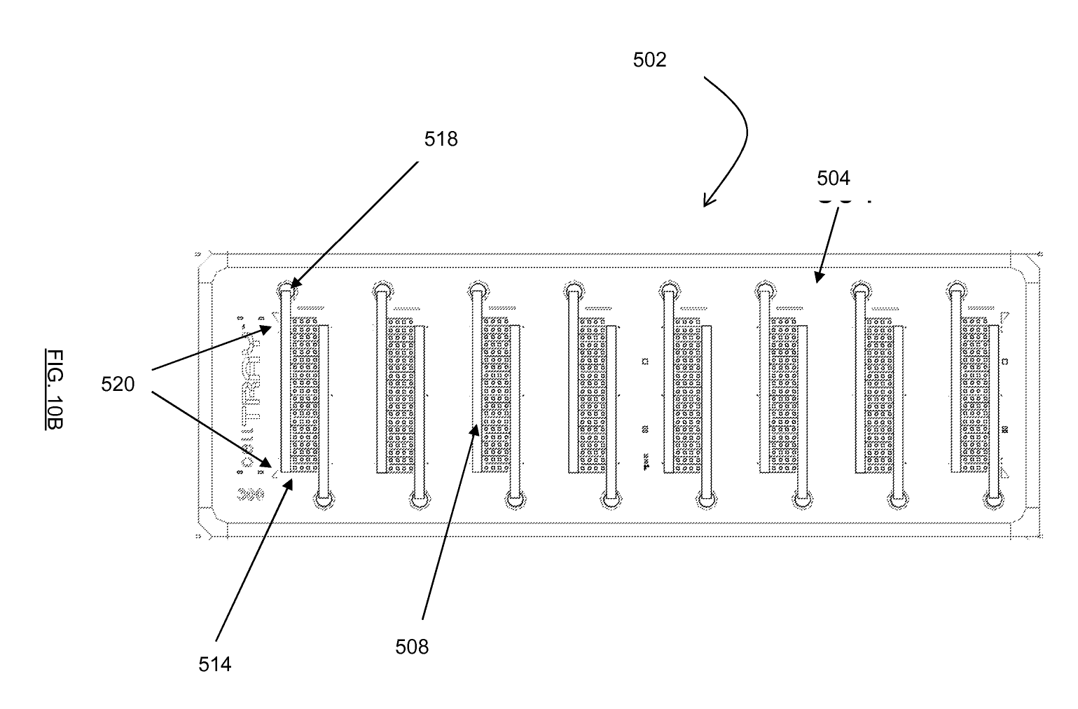

FIG. 10A shows an exemplary embodiment of a cell tray 408 with a set of eight arrays 414 arranged on a substrate 404. As previously described, each array 414 contains a set of cell wells (not shown) that are fed and drained by a set of small microfluidic conduits (not shown). The small microfluidic conduits are in turn fed and drained by a set of large microfluidic channels 408. In the depicted embodiment, there are 8 arrays 414 each containing 132 wells, with each well measuring 200 microns. In the depicted embodiment, the inlets and outlets for the large microfluidic channels 408 are highlighted by visualization markers 418 (e.g., chrome circles) to make them more visible to the naked eye. This facilitates pipetting and other manipulations where the inlets or outlets to the large microfluidic channels 408 are desired to be readily identified. Also depicted in this embodiment is a set of fiducials 420. As described previously, the fiducials may be used to orient the cell tray 402 under the microscope, or to provide other useful information.

FIG. 10B shows an exemplary embodiment of a cell tray having features similar to those shown in FIG. 10A. In the depicted embodiment, a cell tray 502 is shown that has eight cell well arrays 514 arranged on a substrate 504. As previously described, each array 514 contains a set of cell wells (not shown) that are fed and drained by a set of small microfluidic conduits (not shown). The small microfluidic conduits are in turn fed and drained by a set of large microfluidic channels 508. In the depicted embodiment, the inlets and outlets for the large microfluidic channels 508 are highlighted by visualization markers 518 (e.g., chrome circles) to make them more visible to the naked eye. The arrays 514 as shown in this Figure contain larger cell wells than those in FIG. 10A. Each of the 8 arrays 514 for this cell tray 502 contains 80 wells, with each well being 300 microns in size. It would be understood by those of ordinary skill in the art, however, that a cell tray 502 can contain any number of arrays 514 bearing any number of cell wells as would be required for a specific purpose.

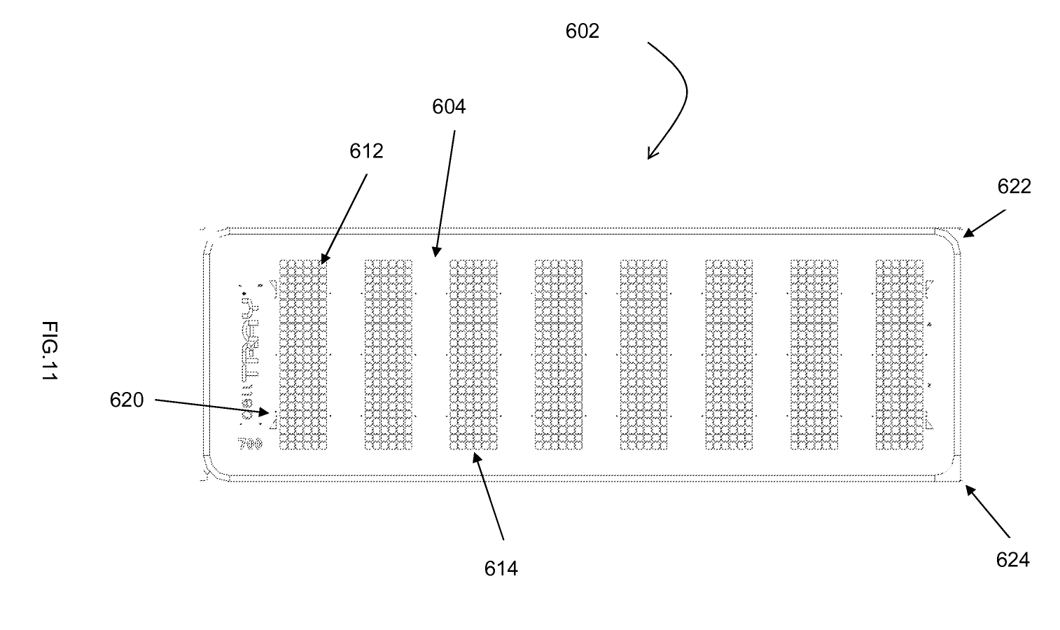

FIG. 11 shows another embodiment of a cell tray 602. In the depicted embodiment, a substrate block 604 is shown that bears a number of cell wells 612 without any interconnecting microfluidics. In the depicted embodiment, there are eight arrays 614, each one containing 144 wells. A cell tray 602 as shown in this figure would be particularly suitable for direct pipetting. In one embodiment, the individual cell wells 612 may be 700 μm wide, with a depth varying from 20 to 500 μm. Other shapes and arrangements of cell wells 612 on a cell tray 602 can be envisioned by those of ordinary skill in the art.

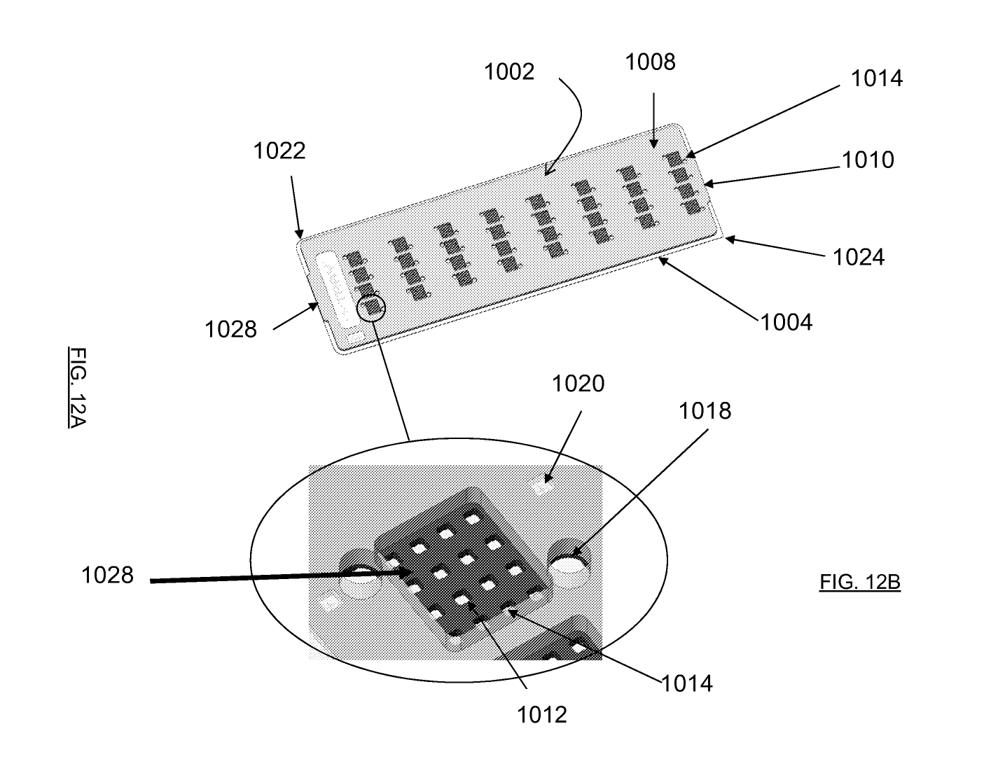

FIG. 12 illustrates yet another exemplary embodiment of a cell tray 1002. In the depicted embodiment, a set of large wells 1014 is positioned as through-holes through a top substrate layer 1008. An exemplary arrangement of these large wells 1014 penetrating the top substrate layer 1008 is shown in FIG. 12A. The cell tray 1002 depicted in this figure is fabricated from three discrete layers. As shown, there is a top substrate layer 1008 bearing 32 large wells (through-holes) 1014. These through-holes may be constructed by etching, for example by direct writing with a CNC laser, or by other methods familiar to those of ordinary skill in the art. A middle substrate layer 1028 bears an array of sixteen wells 1012 corresponding to each large well 1014 in the top layer 1008. The wells 1012 in the middle layer 1028 may be prepared as through-holes traversing the middle layer 1028. These through-holes may correspond to holes that partially penetrate the bottom substrate layer 1004. Alternatively, the through-holes may not penetrate the bottom substrate. The wells 1012 may be fabricated from a photosensitive material, for example by applying a mask, patterned to leave certain regions open to exposure to light; such regions can be readily etched away. One or more fiducials 1020 may be inscribed on the bottom substrate layer 1004 or on the middle substrate layer 1024.

FIG. 12A also illustrates other useful features of the cell tray 1002. In the depicted embodiment, alignment tabs 1028 are formed from the top layer 1010. The cell tray 1002 has a keyed corner 1024 to facilitate alignment, while the other corners 1022 are beveled for easy handling. FIG. 12B shows in more detail an exemplary embodiment of a large cell well 1014 and neighboring structures. The large cell well 1014 contains a number of small cell wells 1012 situated in the middle layer 1028. In some embodiments, the large well measures 2.4 mm×2.7 mm, and the small well measures 16×300 μm. It is understood that other dimensions for large and small wells could be suitable for various purposes. Adjacent to the large well are a set of through holes for air escape 1018. Also depicted in FIG. 12B are two chrome fiducials that may be used for alignment purposes. Other arrangements of fiducials could be substituted as appropriate.

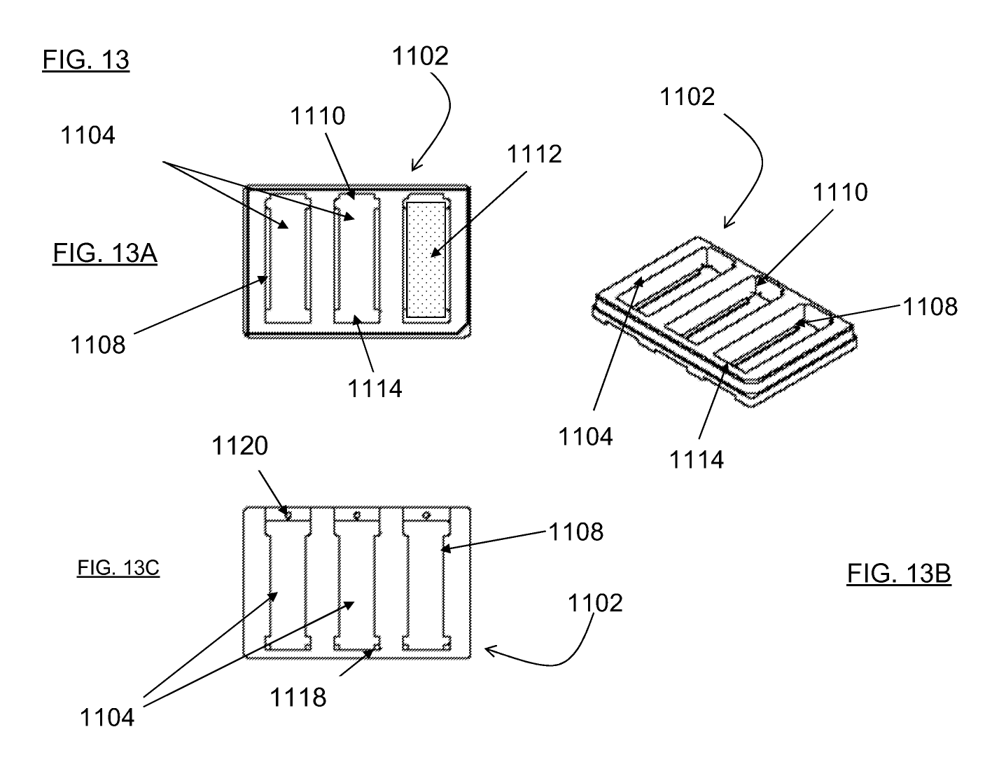

FIG. 13A shows a top view of an exemplary embodiment of a cell tray holder 1102. In the depicted embodiment, there are three container wells 1104, each one of which is configured to hold a cell tray 1112. In the depicted embodiment, the cell tray 1112 may be placed into the container well 1104 from above, and may rest on a set of shelves 1108. In the depicted embodiment, the cell tray 1112 is properly positioned when resting against a backstop 1114 at the back of the container well 1104. So positioned, there may be headroom 1110 in front of the cell tray 1112 at the front of the container well 1104 so that the cell tray 1112 can be conveniently removed. In some embodiments, the cell tray holder 1102 may be fabricated from a black Delrin® plastic, or any other suitable material. The design of the cell tray holder 1102 may be similar to the design of a well plate so that it may be fit into devices that use well plates, such as scanners, microscope stages and the like. FIG. 13B shows a projection of an exemplary embodiment of the cell tray holder 1102 from FIG. 13A, showing the shelf 1108 on the bottom sides of each container well 1104, upon which a cell tray may be supported, and showing the position of the backstop and the headroom. FIG. 13C shows another embodiment of a cell tray holder 1102 with a set of container wells 1104. In the depicted embodiment, each container well contains a set of bumpers 1118 in the back of the container well and a set of shelves 1108 along the sides. In the depicted embodiment, a cell tray may be inserted into the container well 1104 so that it rests on top of the shelves 1108. The bumpers 1118 interface with the superior aspects of the cell tray to hold its corners down. At the front of the container well 1104, a depressible button 1120 holds the front edge of the cell tray in place so that it does not dislocate anteriorly. The button may be made of a resilient material that is compressed when the cell tray is inserted into the container well 1104, or it may comprise a spring, a latch, or the like, that is depressed or engaged as the cell tray is inserted into the container well 1104.

While the invention has been described in connection with certain preferred embodiments, other embodiments would be understood by one of ordinary skill in the art and are encompassed herein.

A method and system for providing and using cell trays have been disclosed. The method and system have been described in accordance with the embodiments shown, and one of ordinary skill in the art will readily recognize that there could be variations to the embodiments, and any variations would be within the spirit and scope of the present application. Accordingly, many modifications may be made by one of ordinary skill in the art without departing from the spirit and scope of the appended claims.

1. A cell tray, comprising
a first layer of an optically transparent substrate material, and
a second layer on top of the first layer, the second layer including a plurality of cell wells, each of the plurality of cell wells being formed by penetrating the second layer to a preselected depth.

2. The cell tray of claim 1 wherein the second layer includes a plurality of through-holes therein, the plurality of through-holes forming a portion of the plurality of cell wells.

3. The cell tray of claim 2 wherein the plurality of cell wells partially penetrate the first layer.

4. The cell tray of claim 1 wherein the second layer includes a plurality of arrays of cell wells.

5. The cell tray of claim 4 wherein each of the plurality of arrays are fluidically isolated from another of the arrays.

6. The cell tray of claim 1 further comprising:
a third layer on top of the second layer, the third layer bearing a through-hole dimensionally corresponding in length and width to dimensions of the array in the second layer, the through-hole being positioned over the array so as to permit access to the array through the through-hole.

7. (canceled)

8. (canceled)

9. The cell tray of claim 1 further comprising:
a microfluidics system to circulate fluid among the cell wells.

10. The cell tray of claim 9 further comprising:
microfluidic channels to provide fluid influx and efflux to the microfluidics system.

11. The cell tray of claim 10 further comprising:
a visualization marker to identify the microfluidic channel.

12. The cell tray of claim 1 wherein the second layer includes a metallic material

13. The cell tray of claim 1 wherein the second layer includes a ceramic material.

14. The cell tray of claim 1 wherein the array includes round cell wells.

15. The cell tray of claim 1 wherein the array includes quadrilateral cell wells.

16. The cell tray of claim 1 wherein the preselected depth is less than a thickness of the second layer.

17. (canceled)

18. The cell tray of claim 1 wherein the first layer bears a fiducial marker.

19. The cell tray of claim 1 wherein the second layer bears a fiducial marker.

20. The cell tray of claim 1 further comprising a registration key.

21. (canceled)

22. The cell tray of claim 6, further comprising:
a gasket on a top surface of the third layer surrounding the through-hole.

23. (canceled)

24. A cell tray, comprising:
an optically transparent substrate material including a plurality of cell holders, the plurality of cell holders arranged in a plurality of arrays, each of the plurality of arrays including cell wells of a preselected dimension, with each of the plurality of arrays being fluidically isolated from another of the plurality of arrays.

25. The cell tray of claim 24 wherein the cell holder is a cell immobilization zone.

26. The cell tray of claim 25 wherein the cell immobilization zone includes a printed microarray of cell-adherent areas.

27. The cell tray of claim 24 wherein the cell holder is a cell well.

28. The cell tray of claim 27, wherein each cell well in an array is fluidically isolated from another cell well in the array.

29. (canceled)

30. The cell tray of 24 further comprising
a microfluidics system that provides fluid communications to the cell wells within the array.

31. The cell tray of claim 30 wherein the microfluidics system interfaces with a plurality of microfluidics channels.

32. The cell tray of claim 31 further comprising:
a visualization marker for identifying the microfluidics channel.

33. (canceled)

34. The cell tray of claim 31 further comprising a sensor to monitor a fluid flow through the microfluidics channel is monitored.

35. (canceled)

36. (canceled)

37. The cell tray system of claim 24 further comprising:
a gasket surrounding the array.

38. (canceled)

39. (canceled)

40. A cell tray system comprising:
a cell tray including at least one of a first cell tray and a second cell tray, the first cell tray including a first layer and a second layer, the first layer including an optically transparent substrate material, the second layer residing on top of the first layer, the second layer including a plurality of cell wells, each the plurality of cell wells being formed by penetrating the second layer to a preselected depth, the second cell tray including an optically transparent substrate material and including a plurality of cell holders, the plurality of cell holders being arranged in a plurality of arrays, each of the plurality of arrays including cell wells of a preselected dimension, each of the plurality of arrays being fluidically isolated from another of the plurality of arrays; and
a cell tray holder bearing container wells dimensionally adapted to contain the cell tray of claim 1 or claim 24.
